(19) United States
(12) Patent Application Publication
(10) Pub. No.: US 2011/0230810 A1
(43) Pub. Date: Sep. 22, 2011

(54) IMPLANTABLE ANTI-ADHESION THREE LAYER PATCH

(76) Inventors: JaiShankar Raman, Chicago, IL (US); Karl R. Leinsing, Hampton, NH (US)

(21) Appl. No.: 12/989,611

(22) PCT Filed: Apr. 23, 2009
(86) PCT No.: PCT/US09/02503

§ 371 Date: Jun. 9, 2011

Related U.S. Application Data
(60) Provisional application No. 61/125,158, filed on Apr. 23, 2008.

(51) Int. Cl. A61F 13/00

(52) U.S. Cl. 602/44; 602/43

(57) ABSTRACT

The present invention relates to a layered membrane sandwich system. In its simplest form, the present invention is a three layer membrane sandwich having a center layer of polyester positioned between first and second layers of silicone. The present invention can be used generally as an anti-adhesion membrane or as a barrier to tissue reaction following surgery. In addition, the present invention may optionally be used as a pericardial patch.

### CROSS REFERENCE TO RELATED APPLICATIONS

This application is a U.S. National Phase application of International Application No. PCT/US2009/002503 filed Apr. 23, 2009, which claims priority to U.S. Provisional Application Ser. No. 61/125,158, filed Apr. 23, 2008, which is incorporated herein in its entirety.

### TECHNICAL FIELD

The present invention relates to a layered membrane sandwich system. In its simplest form, the present invention is a three layer membrane sandwich having a center layer of polyester positioned between first and second layers of silicone. The present invention can be used generally as an anti-adhesion membrane or as a barrier to tissue reaction following surgery. In addition, the present invention may optionally be used as a pericardial patch.

### BACKGROUND OF THE INVENTION

All surgery is bedevilled by the formation of adhesions. Adhesions are fibrous tissue reactions that form as a consequence of tissue handling and can be a major cause of morbidity. The biggest drawback of adhesions is the fact that repeat surgery becomes much more difficult and hazardous. Many materials have been tried, tested and used in vain as anti-adhesion barriers.

What is instead desired is an anti-adhesion barrier that can be applied onto tissue during surgery, but can later be easily removed without damaging the tissue. This problem is especially pronounced when the surgical patch or implant is a long-term implant, such as when it is used as a pericardial patch following cardiac surgery.

Pericardial adhesions after cardiac surgery are common. They may cause hemodynamic issues (Alharthi, M. S. et al., Eur. J. Echocariogr. 10(3): 357-362 (2009).) In addition, these adhesions cause significant problems during reoperations. The extent to which these adhesions cause problems is not fully known, because patients are not commonly imaged with complex echocardiographic techniques or taken back for reoperative surgery. This issue of pericardial adhesions becomes important if these patients are likely to undergo further cardiac surgery or need to have re-evaluation of their mediastinum surgically.

Despite evidence that closing the pericardium preserves right ventricular function and reduces the risk of cardiac injury during subsequent surgery, few surgeons make attempts to close the pericardium. A variety of alternative strategies have been developed to replicate pericardial closure, ranging from mobilizing a pleural fat pad, to making a mesh of the pericardium, to a range of pericardial substitutes. The most common pericardial substitute is expanded polytetrafluoroethylene or “ePTFE” (Goretex Pericardial Membrane, AE Gore & Associates, Flagstaff, Ariz.) which demonstrates good long-term data. However, even this accepted form of therapy has significant issues in terms of underlying tissue distortion, variability of adhesions, etc. (Bunton, R. W., et al., J. Thoracic Cardiovasc. Surg. 100(1): 99-107 (1990).) This large study in sheep evaluated a range of pericardial substitutes and concluded that there was no difference between the substitutes and none of them was better than re-suturing pericardium. However, their recommendations were predominantly for coronary artery bypass graft surgery.

The average cardiac surgeon is now faced with more complex patients who often require combined procedures. In pediatric cardiac surgery, many patients require multiple staged cardiac procedures. Similarly, almost one-third of the heart transplant patients have had at least one previous operation and frequently have undergone Ventricular Assist Device therapy to optimize the patient. There is therefore a significant need for a reliable pericardial substitute.

A variety of polymers have been tested and found to be ineffective in reducing adhesions (Patila, T., et al., J. Surg. Res. 148(2): 181-184 (2008).) There are also reports of various types of silicone-containing systems that have shown reduced adhesion formation as pericardial substitutes, such as Laser Treated Silicone (Amanpour S., et al., J. Long Term Eff. Med. Implants 15(4): 347-354 (2005)); triple layered silicone-urethane co-polymer called UBE (Kajihara N., et al., J. Thorac. Cardiovasc. Surg. 129(1): 18-24 (2005)); and a silicone-coated polyester fabric (Meus, P. J., et al., J. Thorac. Cardiovasc. Surg. 85(1): 54-58 (1983).) However, in all such systems, certain disadvantages were noted, such as severe fibrous epicardial reactions with the silicone-coated polyester fabric.

Accordingly, it would be desirable to improve the anti-adherence and anti-adhesion characteristics of these systems such that they can be removed after a period of time without damaging the underlying cardiac tissues, or damaging any of the surrounding tissues.

Moreover, it would be especially advantageous that such improved devices would not inhibit normal tissue growth, such as occurs in pediatric patients where the heart grows over time. As a result, there is a need for a system that permits heart growth without adhering to (and damaging) the cardiac tissue over time.

It is also desirable that such a novel system be sufficiently strong that it can be sutured (or otherwise anchored) into position without slippage or damage.

### SUMMARY OF INVENTION

In its simplest embodiment, the present invention comprises a layered implantable anti-adhesion device, comprising: a polyester layer disposed between two silicone layers.

In various applications, the device may be formed in the shape of a substantially planar patch, for example, a pericardial patch.

Preferably, the polyester layer is dimensioned to support suturing therethrough.

In alternate embodiments, the patch may be knitted or woven. A knitted patch is more flexible and stretches in more directions than a woven patch. It is to be understood that the present invention is not limited to being knitted or woven and that the present invention encompasses both these designs and alternate designs as well.

In various alternate embodiments, the patch is advantageously made of translucent or semi-opaque materials, such that the vasculature can be visualized through the patch during implantation.

In addition, the patch may be manufactured to be cut from a larger sheet of material. As such, it may be dispensed from a roll and then cut into particular desired sizes before or during a medical procedure.

Accordingly, in one embodiment, the patch of the present invention is an implantable three-layer anti-adhesion patch of a first silicone layer and a second silicone layer, wherein the silicone consists of a silicone polymer; and a polyester layer disposed between the first silicone layer and the second silicone layer.

In one embodiment, the patch is not opaque, i.e. it is translucent or semi-opaque (e.g., less than 70% opaque.)

The polyester may be woven, nonwoven or knitted, and may range in thickness from between 0.015 to 0.020 inches.

Another aspect of the present invention is a method of closing an opening in a section of pericardium, which includes the steps of providing the just-described patch, and securing the patch in place covering the opening, such as with surgical stitches.

In yet another embodiment, the present invention is a method of forming a biocompatible implantable patch including the steps of comprising the steps of forming a first layer of silicone and a second layer of silicone with a layer of polyester disposed between the first layer of silicone and the second layer of silicone in the form of a three-layer composite material; subjecting the three-layer composite material to lateral pressure to mechanically adhere the first silicone layer, the polyester layer and the second silicone layer together to form a stable three-layer composite material; and heating the stable three-layer composite material for a sufficient time and at a sufficient temperature to form stable bonds between layers.

In such methods of manufacture, the first layer of silicone and the second layer of silicone may be applied to the polyester mesch as a liquid form a silicone.

Other aspects of the invention are found throughout the specification.

### BRIEF DESCRIPTION OF THE DRAWINGS

FIG. 1 is a perspective view of a first (multi-purpose) embodiment of the present invention.

### DETAILED DESCRIPTION OF THE DRAWINGS

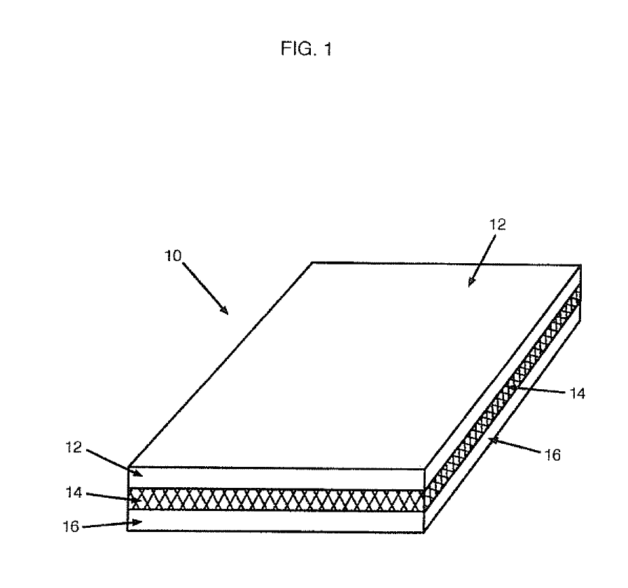

FIG. 1 is an illustration of a first embodiment of the present invention, being a sandwich of silicone layers on either side of a polyester layer. It should also be understood that the silicone layers are adhered to but discreet from the polyester layer, which specifically excludes silicone-coated (i.e., sprayed or dipped) polyester materials that do not provide an adequate deterrent to formation of fibrous adhesions.

In addition, each silicone layer consists of a silicone polymer. As used herein, the term “polymer” means a single-component polymer, as opposed to a co-polymer of silicone and one or more additional materials. In one embodiment, the silicone layers are essentially 100% dimethyl silicone. In particular, co-polymers of silicone and urethane are excluded from the present invention, because urethane does not have sufficient long term strength.

One or both of the silicone layers may also be laser treated, which is known to prevent adhesion formation when used as a pericardial implant (Amanpour, S., et al., J. Long Term Eff. Med. Implants 15(4): 347-354 (2005).) However, the laser treated silicone which is used by itself in such applications is not strong enough for long-term implantation.

Silicone sheeting and precursors thereof can be obtained from a variety of commercial sources, such as Specialty Silicone Fabrications, Inc. (Paso Robles, Calif.)

The polyester layer may be in the form of woven, nonwoven or knitted polyester. In one embodiment, the polyester is woven, and is only “stretchable” in a diagonal direction. Such woven polyester provides a particularly well suited reinforcement layer when the patch is stitched or otherwise anchored in place at the site of implantation.

Specifically, device 10 is a layered implantable anti-adhesion device, comprising: a first silicone layer 12; a second silicone layer 16, and a polyester layer 14 disposed between the first and second silicone layers.

As such, device 10 is a membrane sandwich that exploits the anti-adhesion properties of silicone together with the toughness and durability of polyester to provide a layered system which can be placed against an internal organ or body part, and also used as an anchor for sutures or other surgical attachment means, but can easily be removed without damaging the tissue. Specifically, the outer silicone layers 12 and 16 can be placed against tissues and then removed, doing little damage to the tissues. Thus, device 10 can be easily applied and then pulled off of internal organs as needed.

Accordingly, the non-stick properties of the silicone provide a device that is easily removable without damaging organs. In addition, device 10 is especially useful for pediatric applications (i.e., for long term implants that are placed against tissues that change in shape as the child grows).

In optional embodiments, device 10 is made from a translucent or semi-opaque materials, which means that the device can be placed over the organs or tissues during implantation and the surgeon can still visualize the vasculature underneath. In other optional embodiments, device 10 is made as a roll of material such that a surgeon may cut desired patch sizes from the roll. Alternately, device 10 may be manufactured in small patch sizes (for example, 6×6 inches, 2×3 cm, 7×20 cm, etc.) Moreover, in optional embodiments, device 10 is made from 0.015 to 0.020 inches thick. In one exemplary embodiment, the polyester mesh of layer 14 is 0.004″ thick. It is to be understood that the forgoing dimensions are merely exemplary and are not limiting of the present invention.

In addition, the center polyester layer 14 provides a firm structure that is capable of being secured in place, such as by suturing, while holding device 10 together. Specifically, the polyester layer is dimensioned to support suturing there through without the membrane sandwich falling apart.

In various embodiments, device 10 may be formed in the shape of a patch that is quite inert and easy to handle. The tissue reaction to this material is minimal. Therefore, this material can be easily peeled off underlying structures or removed with the use of electro-cautery. This has tremendous applications in redo or repeat cardiac surgery, surgery of heart requiring insertion of ventricular assist devices, pediatric cardiac surgery (where repeat procedures are likely), surgery for inflammatory bowel disease, etc.

### Uses

The material used to make device 10 is a composite of a layer of polyester between two layers of silicone. There is a large implant experience with silicone in breast implants, penile and scrotal prostheses, etc. However, prior to the current invention, there was no system for a silicone sheet that is used as an anti-adhesion barrier or membrane.

One exemplary use of device 10 is during abdominal surgery, as follows. Typically after abdominal surgery, the abdominal wall is reconstituted and closed in layers. The omentum usually lies immediately underneath the abdominal wall. However, if there is any inflammation or intra-peritoneal infection, omentum works to wall off that area of inflammation. As a consequence, bowel and other abdominal contents can be adherent to the abdominal wall. Adhesions can be a common cause of internal herniation, bowel obstruction and pain following surgery. The use of the present anti-adhesion membrane will dramatically reduce the extent of adhesion formation, and consequently complications from them.

Another exemplary use of device 10 is during heart surgery, as follows. After heart surgery, only a small group of cardiac surgeons make an attempt to close the pericardium, for fear of tamponade (increased pressure on the ventricles) in the short term and pericardial constriction (restriction of ventricular filling by a tightly adherent pericardium) in the long term. As a consequence, adhesions are the norm after any cardiac surgical procedure, making the next procedure quite demanding technically. In addition to obscuring tissue planes, the surface of the heart gets altered, making it difficult to recognize epicardial coronary arteries. Repeat cardiac surgery is also fraught with injury to major cardiac and vascular structures within the chest, due to adhesions of these structures to the chest wall. Adhesions can be a major part of post-operative bleeding in repeat surgery in the setting of heart failure, such as transplantation, explant of ventricular assist devices, endocarditis, etc. In each of these instances, the extra bleeding and treatment with blood products due to the dissection of adhesions is a major cause of increased morbidity, risk and mortality. The use of the present anti-adhesion barrier/membrane system 10 dramatically reduces the likelihood of adhesions and prevents all the consequent complications.

The present device is initially presented to the surgeon as a flattened, flexible device that is easy to handle during an operation.

Moreover, as stated above, the present invention is ideally suited for pediatric use as it does not adhere to organ tissue as the organ grows (as in the case of long-term pediatric patches or other implants).

For use according to the present invention, in one embodiment, the layers of the patch are co-extensive, i.e., each of the three layers extends to all edges of the patch.

### Fabrication

The patch material of the present invention can be fabricated by any known method of forming a multi-layer biocompatible implant. Such methods include, for example, “calendaring”, which involves pushing the three layers (i.e. the two layers of non-vulcanized silicone and the polyester fabric) through a set of rollers under pressure such that they become mechanically adhered to one another. Other methods for forming multi-layered implant materials under pressure are well known.

For example, silicone layers can be applied to polyester fabrics using a “knife-over-roll” procedure, which is suitable for applying thicker layers of silicone. In this method, the blade design can be selected to achieve the desired thickness of the layer. Catalysts such as platinum can also be included to facilitate curing.

Alternatively, the silicone layer can be preformed as a sheet and the polyester layer can be sandwiched between two sheets of silicone under pressure to cause them to adhere together. The term “lateral pressure” as used herein means that there is physical force put upon the sandwich from both sides, as opposed to being subjected to atmospheric overpressures.

After adherence of the three layers together under pressure, in one embodiment, the layers are further enjoined by heating/curing for a sufficient length of time and at a sufficient temperature to achieve the desired performance characteristics. Such conditions can easily be determined by routine optimization. For example, most liquid silicone rubber layers can be cured in 1 to 2 minutes at 160-180° C.

### Experiments

The silicone-polyester sandwich of the present invention was implanted in 24 sheep with rapid pacing induced heart failure. The implant duration ranged from 34 to 263 days (mean of 130 days). At repeat surgery, explant or terminal procedure, the surgeon was able to safely dissect down on to the silicone polyester sandwich using electro-cautery dissection. There was a nice discrete fibrous capsule around the implant with no adhesion of the material to the underlying epicardium or overlying pericardium. This is unlike almost all other pericardial substitutes, which tend to cause either a severe epicardial fibrous reaction, pericardial reaction or both (Ozeren, M., et al., Cardiovasc. Surg. 10(5): 489-493 (2002); and Eng, J., et al, Ann. Thorac. Surg. 48(6): 813-815 (1989).) Furthermore, a re-operative procedure at two separate animal laboratories (North Carolina State University Veterinary School and St. Joseph's Translational Research Institute) showed that there was no distortion of underlying cardiac anatomy.

Histopathology at explant also showed the fibrous capsule had fibers arranged in a parallel fashion which prevented in-growth into the silicone. The reaction of three different approaches to the epicardium and pericardium were also directly contrasted: a) silicone, b) polyester, c) direct apposition of native epicardium to pericardium. The silicone had the least reaction on three counts—findings on reoperation, visual inspection and histopathologic examination. In addition, there were two annuals, who had wound infections and it was striking that there was no infection found on the silicone. The infection seemed to lodge in the polyester implants and in pockets between epicardium and pericardium.

The silicone material was also easily removed at re-operative surgery or necropsy, without injury of the underlying cardiac tissue or overlying pericardium. There was no evidence of increased fibrous tissue reaction in the epicardium where silicone was in direct contact for the duration of the implant. These findings suggest that the silicone-polyester-silicone sandwich design, where the polyester is completely encased between two separate silicone layers, is preferential to a polyester material that is simply sprayed or dipped in silicone, which would not provide an adequate silicone barrier.

The examples set forth above are provided to give those of ordinary skill in the art with a complete disclosure and description of how to make and use the preferred embodiments of the present invention, and are not intended to limit the scope of what the inventors regard as their invention. Modifications of the above-described modes for carrying out the invention that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

We claim:

1. A layered implantable three-layer anti-adhesion patch, comprising:
a first silicone layer and a second silicone layer, wherein the silicone consists of a silicone polymer; and
a polyester layer disposed between the first silicone layer and the second silicone layer.

2. The patch according to claim 1, wherein the patch is not opaque.

3. The patch according to claim 1, wherein the polyester layer is knitted.

4. The patch according to claim 1, wherein the polyester layer is woven.

5. The patch according to claim 1, wherein the patch has a thickness from 0.015 to 0.020 inches.

6. A method of closing an opening in a section of pericardium, comprising the steps of:
providing a patch according to claim 1; and
securing the patch in place covering the opening.

7. The method according to claim 6, wherein securing the patch in place further comprises stitching the patch to the pericardium surrounding the opening.

8. A method of forming a biocompatible implantable patch comprising the steps of:
forming a first layer of silicone and a second layer of silicone with a layer of polyester disposed between the first layer of silicone and the second layer of silicone in the form of a three-layer composite material;
subjecting the three-layer composite material to lateral pressure to mechanically adhere the first silicone layer, the polyester layer and the second silicone layer together to form a stable three-layer composite material; and
heating the stable three-layer composite material for a sufficient time and at a sufficient temperature to form stable bonds between layers.

9. The method according to claim 8, wherein the first layer of silicone and the second layer of silicone are applied to the polyester mesch as a liquid form a silicone.
